(19) United States
(12) Patent Application Publication
(10) Pub. No.: US 2011/0222049 A1
(43) Pub. Date: Sep. 15, 2011

(54) METHOD AND DEVICE FOR TAKING A SAMPLE

(76) Inventor: Kari Seppala, Helsinki (FI)

(21) Appl. No.: 12/672,073

(22) PCT Filed: Aug. 15, 2008
(86) PCT No.: PCT/FI2008/050462

§ 371 Date: Feb. 4, 2010

(30) Foreign Application Priority Data
Aug. 17, 2007 (FI) 20070623

(51) Int. Cl. G01N 33/15

(52) U.S. Cl. 356/51

(57) ABSTRACT

Method and device for taking and measuring a sample of a material of a process, for example, a granulation process. The material to be measured is allowed to accumulate as free fall into the measuring chamber (7) of a measuring means, such as a cuvette (3), the desired measurement is performed through the wall of the measuring means (3) and the measuring means (3) is emptied by means of a pulse of a turbulence-causing, gaseous substance, directed from the bottom upwards in the measuring chamber. (7). The measuring means (3) of the device is divided by a partition (6) into two parts, so that the gas pulse is directed to near to the bottom of the measuring means on one side (5) and from the bottom upwards on the other side (7) of the wall (6).

The present invention relates to a method and apparatus for taking a sample. In particular, but in no way solely, the invention relates to taking a sample from a process, for instance a granulation process used in the manufacture of pharmaceuticals.

It is obvious, without having to be stated separately, that the body of each process is its proper supervision and monitoring during the process, and not simply after it. If something must be done to achieve an excellent result, adjustments can be made immediately, and not only once part of the material produced by the process has already been spoiled due to wrong parameters.

Taking a sample, for example, from a granulation process in the pharmaceutical industry has proven to be problematic. Slowness is one problem. For certain measurements, a sample of the material to be granulated must be taken from the granulation vessel, thus preventing so-called in-line measurement, but instead time is spent taking the sample, transferring it to an external measurement point and performing the measurement there, only after which can fine tuning of the granulation can be performed.

A second problem is that, especially in a granulation process, the adhesion of the material to the measuring cuvette or corresponding device is a fact. Methods have even been developed, in which compressed air is used to fill the measurement device. However, these have turned out to be a poor alternative, because the use of compressed air has usually increased the wrong kind of adhesion of the material to the measuring device. A direct compressed-air jet onto the surface of the window has also been used to clean the window. The jet must be sufficiently powerful for cleaning, but in practice a powerful jet shoots new particles against the surface and, especially if the materials used are adhesive and the granulation liquids binding, material remains on the surface the whole time, soon coating the window and making it useless for measurement. Attempts have also been made to warm the window to prevent adhesion, but the warming usually causes further problems, due to the drying and binding of the substance.

The present invention is intended to create a method and apparatus, with the aid of which the drawbacks plaguing the prior art can be avoided. An additional intention is to create a method and apparatus, with the aid of which measurement is very rapid, measurement can be made as in-line measurement, and, as an additional advantage, an apparatus is created, which does not need cleaning between measurements, but which can be used continuously.

The aforementioned and other benefits and advantages of the present invention are achieved in the manner described as characteristic in the accompanying Claims.

The method according to the invention will be examined in connection with the description of the device. A more detailed description of the apparatus will be made with reference to the accompanying drawings, which show one exemplary apparatus.

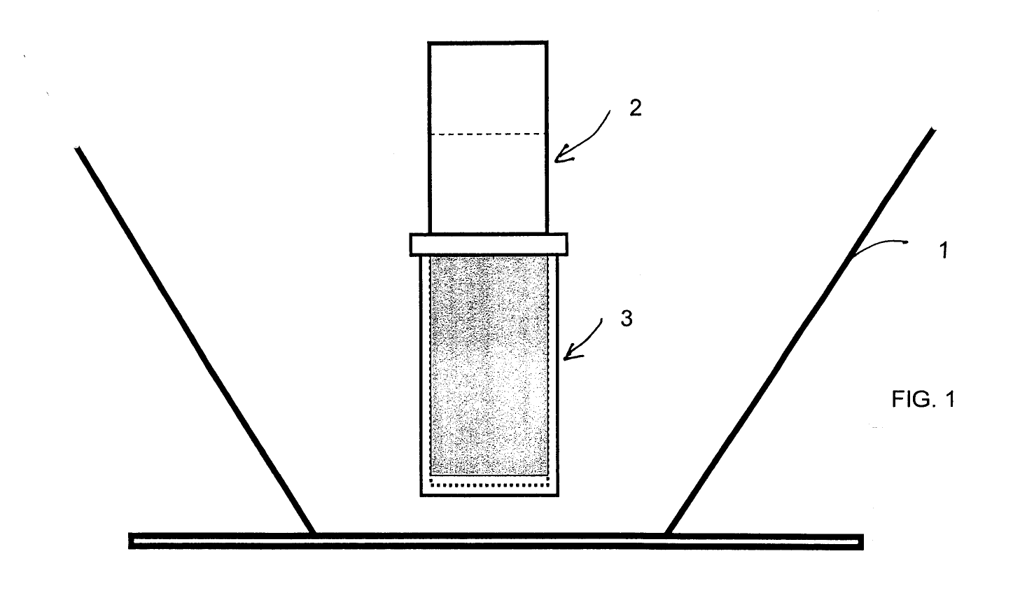

FIG. 1 shows the apparatus according to the invention located in a granulation vessel, and

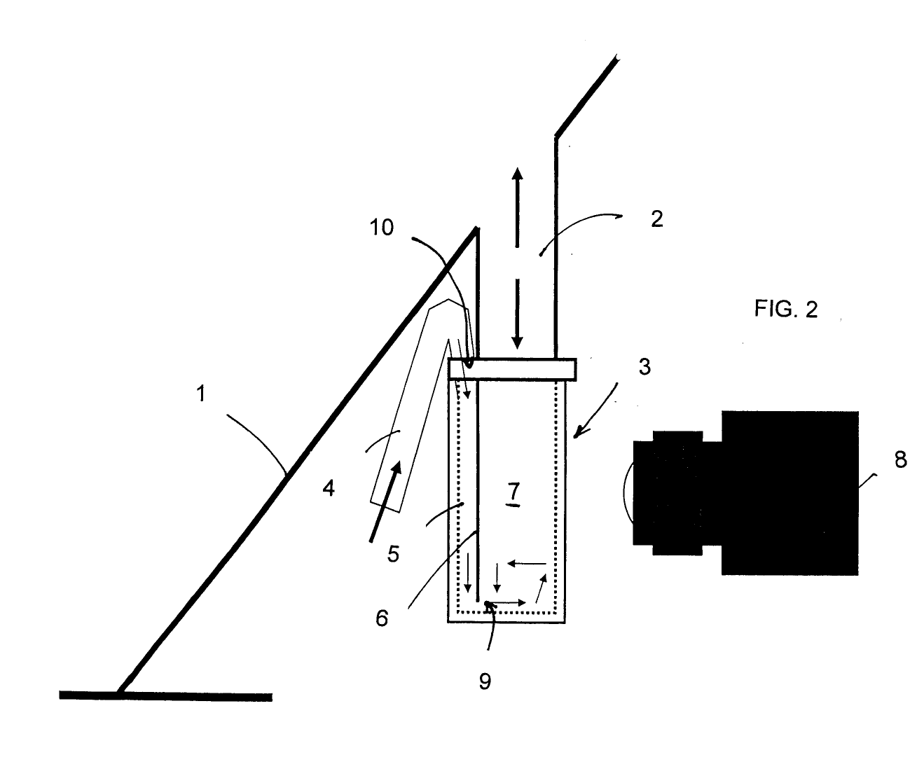

FIG. 2 shows the same apparatus, now seen at an angle of 90 degrees to the former.

It is best to look at FIGS. 1 and 2 placed next to each other, as the marked parts will then easily appear in the correct order.

In the figures, the conical wall of the granulation vessel are marked with the reference number 1. Continuous movement takes place in the material batch to be granulated, while the granulation liquid to be sprayed as an aerosol into the batch becomes effectively mixed onto the surface of the particles to be granulated, increasing their particle size and finally forming granules.

In a traditional process, sampling also interferes with the said granulation-liquid aerosol, which, for understandable reasons, is a mixture of an adhesive kind. In a sampler according to the invention, this problem does not appear.

As can perhaps be seen more clearly from FIG. 2, according to the invention, a shortish vertical channel 2 is made in the sloping wall of the granulation vessel. A measuring cuvette 3 is attached to the lower end of the channel. The joint between the channel 2 and the cuvette 3 is essentially airtight. However, the cuvette 3 is divided vertically into two parts; the actual cuvette part 7 and an air channel 5, divided from it by a wall 6. An air inlet tube 4 is attached to the air channel 5 in an essentially airtight manner. Its inlet opening is marked with the number 10. The actual measuring device is marked generally with the reference number 8.

Once the granulation process has started, the cuvette 3 fills very rapidly with the material being pelletized, through the channel 2, which is an open channel. At this stage, the properties of the accumulated material can be measured using the desired equipment, through the wall of the cuvette.

The type of measurement used is of practically no significance, other than in the sense that measurement of the type is possible at all in these conditions. For example, it is possible to make, for instance, particle-size measurement, or a so-called NIR analysis, the accuracy of which will improve considerably, because the material in the cuvette has a more or less constant density.

Once measurement has taken place, a short pulse of pressurized gas, mainly air, is blown through the air channel 4 and opening 10, to the chamber 5 and through it also into the chamber 7. The air pulse is short, lasting, for example, for only about 0.5 seconds and, thanks to the strongly turbulent flow, does not direct the particles against the wall of the cuvette, but instead completely flushes the measuring chamber 7 of the cuvette of the material batch. As the arrows seek to show, the air pulse causes strong turbulence through the channel 5 and the flow opening 9 and close to the bottom of the cuvette 3 to be measuring chamber 7 and from there naturally to the granulation chamber.

Once the air pulse has stopped, the channel 2 and the cuvette 3 are again ready to receive material from the granulation chamber. Indeed, the cuvette fills immediately with new material, the properties of which will have altered due to the progress in granulation. Measurement and the emptying and cleaning of the cuvette can be performed immediately.

For example, leading the air pulse to the lower part of the cuvette 3, in such a way that a strong turbulent flow is created, can be performed in some way other than that described above. A channel/opening, which does not run through the part 5 separated from the cuvette 3 by the partition 6, but is lead to the lower part of the measuring chamber 7 in some other manner, can be used to guide the air. The means used can be a separate tube, independent of the cuvette, but connected to it, the flow lead through which is strongly turbulent. The tube can be connected to the cuvette through either its wall or its bottom. One way to create a turbulent flow is to expand the end of the tube near to the cuvette. Of course, many other ways to create turbulence can be easily applied from other fields of technology. If the cuvette is not divided into two parts by a partition, it is possible to perform so-called permeation measurement through the cuvette, because the air channel does not prevent measurement of this kind.

The cuvette can also be of the through-flow type, in which case the cuvette will be open at both ends. Emptying will then be through the lower part of the cuvette, which is connected back to the granulation vessel and is equipped with a compressed-air ejector. In this case, turbulence is created using a nozzle in the upper part of the cuvette and the turbulence flow is directed downwards.

Practical test have demonstrated the functionality of the invention. Measurements are quick and accurate. One important factor in the advantageousness of the invention is that the cuvette is cleaned extremely well with the air pulse used. This appears to be at least mostly due to the fact that the material coming to the cuvette has not caked due to some external factor and the accumulation of the material takes place as free fall. In addition, the accumulation stage is not assisted with air pulses, as is done in some known systems. In these cases, there is the danger, as stated earlier, of the material adhering strongly to the surfaces. A short air pulse will also not dry the material.

The measuring cuvette can also be disposable. The measuring cuvette can also be surfaced with a suitable coating, in order to hinder the adhesion of the substance. In certain conditions, this will help the surfaces of the cuvette to remain clean.

The method according to the invention is stable in many ways. The measuring cuvette is in the same conditions in terms of humidity and temperature as the granulation vessel, thus avoiding problems arising from differing conditions. The method also functions when difficult binding granulation liquids are used. Because the cuvette fills very rapidly after emptying, under no circumstances does the granulation liquid as such enter the cuvette, but only attached to the surface of the pellets.

The invention is described above in connection with one functional totality. However, it is obvious that the invention can be varied in many ways, while nevertheless remaining within the scope of the protection of the inventive idea and the accompanying Claims.

1. Method for taking and measuring a sample in a process, for example, the material of a granulation process, in which the material is allowed to accumulate in a measuring means, and the desired measurement is performed from the measuring chamber (7) of the measuring means (3), after which measurement the measuring means (3) is emptied with the aid of a pulse of turbulence-causing, gaseous substance directed into the measuring chamber (7), characterized in that the collection of the sample is performed through a channel (2) leading from the wall plane of the process vessel (1) to a measuring chamber (7) outside the vessel.

2. Method according claim 1, characterized in that the measuring means (3) is a cuvette, through the wall of which the measurement is performed.

3. Method according to claim 1, characterized in that the material to be measured is collected in free fall to the cuvette (3) and that the emptying pulse is guided to the lower part of the measuring chamber.

4. Method according to claim 1, characterized in that the measuring means (3) is divided into two parts by a partition (6), in which case the gas pulse is directed close the bottom of the measuring means on one side (5) and from the bottom by the other side (7) of the wall (6).

5. Method according to claim 1, characterized in that a short pulse of pressurized air is used.

6. Method according to any of the above claims, characterized in that the pulse of gaseous substance is led to the chamber (5) through an opening (10) in its upper part and to the chamber (7) through a flow opening (9) under the partition (6).

7. Method according to any of the above claims, characterized in that a strongly turbulent air pulse is led to the measuring chamber (7) through a tubular channel penetrating its wall or bottom.

8. Device for taking and measuring a sample of the material of a process, for example a granulation process, in which the material is allowed to accumulate in a measuring means, and the desired measurement is performed from the measuring chamber (7) of the measuring means (3), after which measurement the measuring means (3) is emptied by means of a turbulence-causing, gaseous substance, directed to the measuring chamber (7), characterized in that in the device there is an essentially vertical channel (2), starting essentially from the wall of the process chamber and leading to the measuring means (3), for leading the sample to the measuring means (3) external to the vessel (1).

9. Device according to claim 8, characterized in that the measuring means is a cuvette (3), in which there is a measuring chamber (7) and a channel (5) for bringing the emptying pulse, so that the channel (5) is separated from the chamber (7) by a partition (6), beneath which there is a flow opening (9) for the gas.

10. Device according to claim 9, characterized in that the channel (2) and the cuvette (3) are essentially airtightly connected to each other.

11. Device according to claim 8, characterized in that the cuvette's (3) inlet opening (10) for the gaseous emptying pulse is located in the upper part of the cuvette and opens into the chamber (5) behind the partition (10).

12. Device according to any of the above claims 8-11, characterized in that it comprises a separate tubular channel, which is connected to the chamber (7) through its wall or bottom, for bringing the emptying pulse.

13. Device according to claim 9, characterized in that the cuvette is a through-flow cuvette.

14. Device according to claim 8, characterized in that the measuring device is surfaced with a substance preventing the material being measured from adhering.
